(19) United States
(12) Patent Application Publication
(10) Pub. No.: US 2007/0213562 A1
(43) Pub. Date: Sep. 13, 2007

(54) Recycling process for preparing sertraline

(76) Inventors: Ami Zoran, Lehavim (IL); Ehud Amir, Ramat-Aviv (IL); Ziv Kurgan, Har-Adar (IL); Maxim Karlik, Beer-Sheva (IL); Leonid Metsger, Beer-Sheva (IL)

Correspondence Address: KENYON & KENYON LLP, ONE BROADWAY, NEW YORK, NY, 10004, US

(21) Appl. No.: 11/657,297

(22) Filed: Jan. 23, 2007

Related U.S. Application Data
(60) Provisional application No. 60/761,563, filed on Jan. 23, 2006.
(60) Provisional application No. 60/778,647, filed on Mar. 2, 2006.

(51) Int. Cl. C07C 209/68

(52) U.S. Cl. 564/460

(57) ABSTRACT

Provided is a recycling process for preparing sertraline, which may be carried out on an industrial scale.

### RELATED APPLICATIONS

This application claims the benefit of priority to U.S. Provisional Application Nos. 60/761,563, filed Jan. 23, 2006; and 60/778,647, filed Mar. 2, 2006, the contents of which are incorporated herein by reference.

### FIELD OF THE INVENTION

The present invention relates to recycling processes for preparation of sertraline free of impurities and other stereoisomers.

### BACKGROUND OF THE INVENTION

Sertraline hydrochloride, (1S-cis)-4-(3,4 dichlorophenyl)-1,2,3,4-tetrahydro-N-methyl-1-naphthalenamine hydrochloride, having the formula: 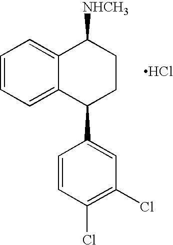
is approved, under the trademark Zoloft®, by the U.S. Food and Drug Administration, as a serotonin re-uptake inhibitor for the treatment of depression, obsessive-compulsive disorder, panic disorder and post-traumatic disorder. Only S-cis sertraline is therapeutically active.

U.S. Pat. No. 4,536,518 (“518 patent”) describes a synthesis of sertraline hydrochloride from sertralone having the following formula:

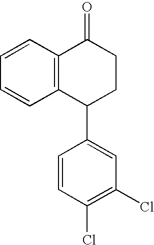

The process for synthesizing sertraline from sertralone has two steps. First, sertralone is condensed with methyl amine in the presence of an acid catalyst, to yield the Schiff base of sertralone, “sertraline-1-imine”:

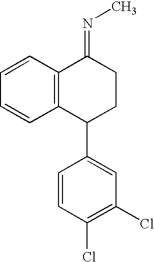

The imine is then reduced to sertraline. The reduction process of the '518 patent involves the hydrogenation of sertraline-1-imine concentrate at room temperature for two hours over 10% Pd/C catalyst in an atmosphere of hydrogen (1 atm pressure). The product is a racemic mixture of the cis and trans diastereoisomers (“(±)-cis/trans-sertraline”) in the ratio of approximately 3 to 1. The '518 patent discloses that reduction with NaBH₄ gives a cis:trans ratio of about 1:1.

As illustrated in the process of the '518 patent, a problem with synthesis of sertraline is the formation of useless stereoisomers which decrease the overall yield. Due to presence of two chiral centers, at C-1 and C-4 (C-4 refers to the carbon to which the dichlorophenyl group is attached), four different stereoisomers are produced during the synthesis process. Of these, one is sertraline (cis-1S, 4S), and its enantiomer (cis-1R, 4R). The other are trans stereoisomers (1R, 4S) and (1S, 4R). Also, the isolation of the product from the isomers mixture is complicated.

Sertraline may be isolated in two ways from the other stereoisomers. Isolation of sertraline from its trans stereoisomers may be done in a classical way by crystallization since distereoisomers have substantially different characteristics. Isolation of sertraline from its enantiomer 1R,4R-Sertraline may be done by selective precipitation with a chiral entity such as mandelic acid.

U.S. Pat. No. 5,082,970, according to its abstract, discloses a process for converting trans-isomeric sertraline to cis-isomeric sertraline by contacting trans-sertraline, or a mixture of same with up to about an equal part by weight of the corresponding cis-isomer, with a basic equilibration agent like potassium t-butoxide in a reaction-inert polar organic solvent to ultimately afford a cis/trans-mixture wherein the amount of cis-amine present in the mixture achieves a constant value of about 2:1 on a weight-by-weight basis.

WO 01/49638, according to its abstract, discloses a process for converting the cis (1R, 4R), trans (1S, 4R), and trans (1R, 4S) stereoisomers of sertraline into sertraline, starting with an initial reaction mixture which contains at least one of these stereoisomers and converting the sertraline stereoisomers into an imine form of sertraline. The imine form of sertraline is then reduced so that sertraline and at least one sertraline stereoisomer byproduct is produced in the reaction mixture. The sertraline is then recovered from the reaction mixture, e.g., by fractional crystallization (followed by resolution of sertraline from the cis (1R, 4R) stereoisomer, if necessary). The reaction mixture is then recycled through the same steps so that sertraline is produced from its stereoisomers in an asymptotic yield. The reaction scheme of WO 01/49638 requires conversion to an imine before isomerization.

WO 97/21662, according to its abstract, provides a process for racemising an optically-enriched chiral amine of the formula: R1-CH(NR3R4)-R2, wherein R₁ is aromatic or unsaturated alkyl; R₂ is aromatic or alkyl; and R₃ and R₄ are independently selected from hydrogen, alkyl and aryl; and wherein any combination(s) of two of the R groups may form a ring; comprises treatment of the optically-enriched amine with a metal hydroxide in an aprotic polar solvent. However, the present Applicants could not isomerize sertraline stereoisomers when carrying out the process of WO 97/21662.

A recycling process for preparation of sertraline is also provided in WO 05/023752.

There is a need in the art for recycling processes which produce sertraline with higher yield, greater purity, which is also economical and applicable on an industrial scale.

### SUMMARY OF THE INVENTION

In one embodiment, the present invention provides an isomerization process comprising isomerization of a mixture having at least one 4R sertraline isomer in the presence of a base and a catalytic amount of a solvent selected from: DMSO, tetrahydrofuran, methyl t-butyl ether, dioxane, sulfolane, N-methyl pyrrolidone and mixtures thereof. This isomerization increases the 4S to 4R ratio.

In another embodiment, the present invention provides an isomerization process comprising isomerizing a mixture having at least one 4R sertraline isomer in presence of a solvent selected from: DMSO, tetrahydrofuran, methyl t-butyl ether, dioxane, sulfolane, N-methyl pyrrolidone and mixtures thereof and a catalytic amount of a inorganic base.

In another embodiment, the present invention provides an oxidation process comprising combining isomerized mixture of sertraline stereoisomers, a mixture of C₁-C₄ alcohol and an alkyl aromatic hydrocarbon, and an oxidizing agent in the presence of a base. The oxidizing agent and the base can be in the form of a complex.

### DETAILED DESCRIPTION OF THE INVENTION

As used herein, the term “catalytic amount” when referring to a solvent, relates to 0.02 to about 2.0 ml/gr compared to the sertraline stereoisomers mixture.

As used herein, the term “catalytic amount” when referring to a base, relates to 0.02 to about 0.5 equivalents compared to the sertraline stereoisomers mixture.

The present invention relates to a process for recycling sertraline which uses a catalytic amount of solvents during isomerization and to a process for recycling sertraline which uses a catalytic amount of a base. The use of a catalytic amount of solvent, and/or a catalytic amount of a base, provides better yield, improved capacity and also eliminates the need of isomerization with large amounts of the base or the solvent.

The process of the present invention comprises isomerization of a mixture having at least one 4R sertraline isomer in the presence of a base and a catalytic amount of a solvent selected from: DMSO, tetrahydrofuran, methyl t-butyl ether, dioxane, sulfolane, N-methyl pyrrolidone and mixtures thereof. This isomerization increases the 4S to 4R ratio. By using a catalytic amount of the organic solvent, there is no need for concentration steps in order to eliminate the organic solvent.

Preferably, the solvent is added in an amount of about 0.02 ml/gr to about 0.2 ml/gr of the sertraline stereoisomers mixture, and more preferably in an amount of about 0.05 ml/gr to about 0.2 ml/gr of the sertraline stereoisomers mixture.

Preferably, the base is an inorganic base. Preferably, the inorganic base is selected from the group consisting of: an alkali metal, alkaline earth metal hydroxide, and a carbonate. Preferably, the alkaline earth metal hydroxide is selected from the group consisting of: NaOH, KOH, LiOH and Mg(OH)₂. Preferably, the alkali metal is selected from the group consisting of: Na, K, Li, Mg, Ca and Ba. Preferably, the carbonate is selected from the group consisting of: sodium carbonate, potassium carbonate, sodium hydrogen carbonate, and potassium hydrogen carbonate. More preferably the inorganic base is NaOH. Most preferably, the inorganic base is KOH. An inorganic base is cheaper than an organic base, and is easier to remove, which renders it more efficient in industrial processes.

Preferably, the base is added in a catalytic amount. More preferably, the base is added in an amount of about 0.1 to about 0.5 equivalents of the sertraline stereoisomers mixture. Most preferably, the base is added in about 0.27 equivalents of the sertraline stereoisomers mixture.

Preferably, the isomerization step is performed at a temperature of about 80° C. to about 150° C., more preferably about 100° C. to about 140° C. Most preferably, the isomerization step is performed at a temperature of about 110° C. to about 135° C.

The isomerization process preferably results in a ratio of about 1.1 to about 1.50 4R to 4S. The reduction in amount of 4R isomer is preferably from at least about 8 to about 1.5 more preferably about 11 to about 1.1 in relation to the 4S isomer.

The process of the present invention further comprises isomerization of a mixture having at least one 4R sertraline isomer using a solvent selected from: DMSO, tetrahydrofuran, methyl t-butyl ether, dioxane, sulfolane, N-methyl pyrrolidone and mixtures thereof, in the presence of a catalytic amount of a base.

Preferably, the base is added in an amount about 0.1 to about 0.5 equivalents of the sertraline stereoisomers mixture. Most preferably, the base is added in an amount of about 0.27 equivalents of the sertraline stereoisomers mixture.

Preferably, the solvent and the base are as described above. Preferably, the solvent is added in a catalytic amount. Preferably, the temperature is as described above.

The mixture of sertraline stereoisomers which may contain (1R, 4R), (1S, 4S), (1S, 4R), and (1R, 4S) provided for the isomerization step also contains sertralone and mandelic acid, which may be eliminated by the methods such as those described in PCT publication No. WO 2005/023752 (counterpart of US 2005/0107636, incorporated herein by reference). Sertralone may be removed by extraction with a water immiscible solvent. The mandelic acid may be removed by extraction at acidic pH with a water immiscible solvent.

The obtained mixture of sertraline and its isomer(s) used in the isomerization process is typically obtained by conversion of sertralone to sertraline-1-imine, followed by hydrogenation of the imine. The total yield of sertraline depends in a great measure on the formation ratio of other stereoisomers. Starting from racemic sertralone, the sertraline enantiomer (1R,4R) inherently is formed in the same ratio as sertraline. Most of the sertraline may be recovered by precipitation with optically pure mandelic acid, but the formation of the other isomers reduces the total yield. The recovery of sertraline isomers obtained together with sertraline remaining after selective precipitation is an important economic aspect of a process for producing sertraline. If optically enriched sertralone is used, it is possible to also end up with pure 1R,4R sertraline.

The isomerized mixture obtained by the above processes may further be oxidized and converted to sertraline. Preferably, the conversion comprises: oxidizing the isomerized mixture to obtain an imine or a ketone at the C1 position; optionally, transforming the ketone to an imine, when the oxidation product is a ketone; and hydrogenating the imine to a mixture of 4 stereoisomers. Oxidation may be carried using a hypohalite or an oxidizing agent such as Br₂ in the presence of a base in a suitable protic solvent. See e.g., WO 2005/023752. Hydrogenation may be carried out according to methods disclosed in WO 98/27050, WO 01/16089, WO 99/57093 or WO 04/092110, incorporated herein by reference. After hydrogenation, the four stereoisomers are produced. Sertraline may be separated by fractional crystallization with a chiral precipitating agent such as mandelic acid, while the other stereoisomers may be recycled.

One of the steps in the recycling process of sertraline includes oxidation of the isomerized mixture of sertraline stereoisomers to obtain an imine or a ketone. The present invention provides an oxidation process in presence of a mixture of C₁-C₄ alcohol and alkyl aromatic hydrocarbon. The use of alkyl aromatic hydrocarbon, particularly toluene, provides higher efficacy, yield and chemical purity. The process comprises: combining isomerized mixture of sertraline stereoisomers, a mixture of C₁-C₄ alcohol and an alkyl aromatic hydrocarbon, and an oxidizing agent in the presence of a base.

The isomerized mixture of sertraline stereoisomers may be obtained by any method known in the art, such as described in this invention or in WO 2005/023752.

Preferably, the C₁-C₄ alcohol is methanol. Preferably, the alkyl aromatic hydrocarbon is toluene. Preferably, the mixture contains about 5 to about 0.5 volume/volume methanol to toluene.

Preferably, the oxidizing agent is selected from the group consisting of: KMnO₄, Br₂ and hypohalite, such as hypochloride.

Preferably, the base is an inorganic base. Preferably, the inorganic base is selected from the group consisting of: an alkali metal, alkaline earth metal hydroxide, and a carbonate. Preferably, the alkaline earth metal hydroxide is selected from the group consisting of: NaOH, KOH, LiOH and Mg(OH)₂. Preferably, the alkali metal is selected from the group consisting of: Na, K, Li, Mg, Ca and Ba. Preferably, the carbonate is selected from the group consisting of: sodium carbonate, potassium carbonate, sodium hydrogen carbonate, and potassium hydrogen carbonate. More preferably the inorganic base is NaOH. Most preferably, the inorganic base is KOH.

After combining the isomerized mixture of sertraline stereoisomers, a mixture of C₁-C₄ alcohol and an alkyl aromatic hydrocarbon, and an oxidizing agent in the presence of a base, a slurry is obtained. The slurry is further maintained for about 0.5 to about 12 hours.

The imine or the ketone obtained from the oxidation process may then be recovered by conventional techniques such as filtration. The imine or the ketone may be re-slurried in water, and then dried. Drying may be carried out under reduced pressure or elevated temperature to accelerate the process. Drying may be carried out for example at a temperature of about 40° C. to about 70° C., preferably at about 45° C. to about 60° C., at a pressure of less than about 100 mmHg. The crude imine or ketone may be further purified by dissolving it in toluene at elevated temperature, such as about 75 to about 85° C., cooling the solution to precipitate the imine or the ketone, such as to a temperature of about −10° C. to about 25° C., preferably at about −7° C. to about 5° C., and recovering the imine. The recovered imine or ketone may be dried such as a temperature of about 45° C. to about 80° C., preferably at about 50° C. to about 65° C., at a pressure of less than about 100 mmHg.

Optionally, the isomerized mixture is diluted with toluene, cooled, such as to a temperature of less than about 20° C. to obtain a solution. A phase transfer catalyst is then added to the reaction mixture. An example of a phase transfer catalyst is tricaprylmethylammonium chloride. A mixture of aqueous NaOCl and NaOH is added to the toluene while maintaining the temperature. The crude imine may then be recovered from the reaction mixture and purified as described above.

The oxidized mixture obtained by the above processes may further be converted to sertraline. After oxidation, the ketone may be transformed to the imine, and the imine is hydrogenated to a mixture of 4 stereoisomers. Hydrogenation may be carried out according to methods disclosed in WO 98/27050, WO 01/16089, WO 99/57093 or WO 04/092110, incorporated herein by reference. Optionally, a cobalt containing catalyst is used for reduction, in a batch reactor or a trickel bed reactor.

After hydrogenation, the four stereoisomers are produced. Sertraline may be separated by fractional crystallization with a chiral precipitating agent such as mandelic acid, while the other stereoisomers may be recycled as disclosed above. Such fractional crystallization may be carried out with or without separation of cis isomers from trans isomers beforehand.

The sertraline produced by the process of the present invention may also be converted to its corresponding salt, preferably to its hydrochloride salt. The sertraline may be converted by contact with gaseous or aqueous HCl. In one embodiment, sertraline is converted into sertraline hydrochloride by a process comprising: dissolving sertraline in an organic solvent to form a solution, adding hydrogen chloride to the solution, and isolating sertraline hydrochloride. In one embodiment sertraline HCl Form II is prepared according to the process set out in U.S. Pat. No. 6,897,340.

Sertraline hydrochloride may be used in pharmaceutical compositions that are particularly useful for the treatment of depression, obesity, chemical dependencies or addictions, premature ejaculation, obsessive-compulsive disorder and panic disorder. Such compositions comprise sertraline hydrochloride and at least one pharmaceutically acceptable carrier and/or excipient known to one of skill in the art.

For example, these pharmaceutical compositions of sertraline hydrochloride may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups, and suspensions. Suitable forms of parenteral administration include an aqueous or non-aqueous solution or emulsion, while for rectal administration suitable forms for administration include suppositories with hydrophilic or hydrophobic vehicle. Suitable forms of topical administration include transdermal delivery systems known in the art. Suitable forms of nasal delivery include aerosol delivery systems known in the art.

Suitable, non-toxic, pharmaceutically acceptable carriers and/or excipients will be apparent to those skilled in the art of pharmaceutical formulation, and are discussed in detail in the text entitled Remington's Pharmaceutical Science, 17^{th} Edition (1985), the contents of which are herein incorporated by reference.

### EXAMPLES

Impurity Profile of Sertraline Hydrochloride by HPLC

HPLC

| | |
|---|---|
| Column & packing: | Chiradex 250-4, 5μ, Merck 1.51333.0001 |
| Eluent: | 52% Ammonium dihydrogen phosphate |
| | 0.05M adjusted to pH 4.2 with H₃PO₄ |
| | 48% Methanol |
| Flow rate: | 1.0 ml/min. |
| Detector: | 220 nm. |
| Sample volume: | 20 μl. |
| Diluent: | Eluent |
| Oven temperature: | 30° C. |

Purity Profile of Sertralone by GC

GC

| | |
|---|---|
| Column & packing: | BP 5, 30 m × 0.53 mm × 1.0μ, SGE |
| | PN 054195 or equivalent column |
| Injector temperature: | 250° C. |
| Detector temperature: | 300° C. |
| Oven temperature: | 220° C. for 10 minutes then increase 10° C./ |
| | minute up to 250° C. for 3 minutes. |
| Injection volume: | 1 μl. |
| Flow: | 14 ml/min Helium |
| Detector: | FID |
| Diluent: | Hexane |

Purity Profile of Imine by GC

| | |
|---|---|
| Column & packing: | BP 5, 30 m × 0.53 mm × 1.0μ, SGE |
| | PN 054195 or equivalent column |
| Injector temperature: | 160-180° C. |
| Detector temperature: | 300° C. |
| Oven temperature: | 220° C. for 10 minutes then 10° C./ |
| | minute up to 250° C. for 7 minutes. |
| Injection volume: | 1 μl. |
| Flow: | 14 ml/min Helium |
| Detector: | FID |
| Diluent: | Diethyl ether |

### Example 1

Step 1: D-Mandelic Acid Recovery

500 grams of mother liquid (toluene) containing 15.0 grams R,R-sertraline mandelate (derived from the separation of S,S-sertraline-base from its racemic mixture by D-mandelic acid treatment, as described in Example 1 of WO 2005/023752) were combined with 25 grams 16% NaOH solution to form a bi-phasic solution. The two phases were heated to 70° C. and mixed for 0.5-1.0 hr. The aqueous phase was separated, washed twice with 20 ml fresh toluene and gradually acidified with 32% HCl (0.3/1.0 w/w) to pH<0.5. The formed D-mandelic slurry was cooled to 0±5° C., filtered by suction and washed 3 times with 20 ml cold toluene. The wet cake was dried at 50° C. for 6 hr under vacuum to yield 3.6 grams of D-mandelic acid (70% yield; 96.5% purity).

Step 2: Isomerization of R,R Sertraline Base

A 1000 ml glass reactor was charged with 100 grams R,R sertraline base (produced by evaporating the toluenic mother liquid after the (+) sertraline-mandelate filtration according to Example 2 of WO 2005/023752), 5.0 grams KOH and 10.0 ml DMSO. The stirred suspension was heated to 130° C. and maintained as a black, homogeneous mass at said temperature for 2-3 hours. A ratio of 4R/4S=1.1 was obtained in the reaction mixture, which was cooled to 50° C.

Step 3: Preparation of “Imine” by Oxidation of Isomerized RR Sertraline Mixture

A. Bromine-NaOH

The isomerized mixture obtained in step 1 was diluted with 50 ml toluene and 500 ml methanol and cooled to 25° C. 40 grams of solid NaOH were gradually added to the mixture, while the temperature was maintained below 60° C. The solution was cooled to 25° C. and 19.0 ml Br₂ was then added dropwise, while the temperature was maintained at 25-35° C. The precipitated imine slurry was maintained at 25° C. for 30 minutes and filtered. The imine was re-slurried in 200 ml of water at 25° C. for 15-30 minutes, filtered and washed twice with 100 ml water. The wet cake was then dried for 2-6 hr at 50° C. under vacuum yielding 70 grams of dry material (93% purity). The dry crude imine was then purified by solubilizing in 140 ml toluene at 75-85° C. for 10-30 min, cooling to 0° C., and filtering at said temperature. The cake was washed with 35 ml of chilled toluene (−5° C.) and dried for 2-6 hr at 50° C. under vacuum yielding 56 grams dry material (97% purity; 54% yield).

B. Aqueous NaOCl

The isomerized mixture obtained in step 1 was diluted with 100 ml toluene, cooled to <15° C. 5 grams Aliquat 336® (Tricaprylmethylammonium chloride) was then added to the isomerized mixture. A mixture of 450 grams aqueous NaOCl (11%) and 40 grams solid NaOH were gradually added to the toluene solution while the temperature of the mixture was maintained at <20° C. The two phase mixture was heated to 25° C., mixed intensively for 0.5 hr and 100 ml were added. The mixture slurry was filtered and the cake washed twice with 100 ml water at 25° C. The wet crude imine (80-90 grams) was then subjected to the purification procedure described in step 2A.

### Example 2

Isomerization of R,R Sertraline Base

A 1000 ml glass reactor was charged with 100 gr RR Sertraline base (produced by evaporating the toluenic mother liquid after the (+) sertraline-mandelate filtration according to Example 2 of WO 2005/023752), 5.0 gr KOH and 10.0 ml DMSO. The stirred suspension was heated to 130° C. and maintained as a black, homogeneous mass at said temp. for 2-3 hr. A ratio of 4R/4S=1.1 was monitored in the reaction mixture which was cooled to 50° C.

### Example 3

Isomerization of R,R Sertraline Base

A 1000 ml glass reactor was charged with 100 gr RR Sertraline base (produced by evaporating the toluenic mother liquid after the (+) sertraline-mandelate filtration according to Example 2 of WO 2005/023752), 5.0 gr KOH and 5.0 ml DMSO. The stirred suspension was heated to 130° C. and maintained as a black, homogeneous mass at said temp. for 2-3 hr. A ratio of 4R/4S=1.17 was monitored in the reaction mixture which was cooled to 50° C.

### Example 4

Isomerization of R,R Sertraline Base

A 1000 ml glass reactor was charged with 100 gr RR Sertraline base (produced by evaporating the toluenic mother liquid after the (+) sertraline-mandelate filtration according to Example 2 of WO 2005/023752), 10.0 gr KOH and 5.0 ml DMSO. The stirred suspension was heated to 130° C. and maintained as a black, homogeneous mass at said temp. for 2-3 hr. A ratio of 4R/4S=1.19 was monitored in the reaction mixture which was cooled to 50° C.

Having thus described the invention with reference to particular preferred embodiments and illustrative Example, those in the art will readily appreciate modifications to the invention as described and illustrated that do not depart from the spirit and scope of the invention as disclosed in the specification. The Example is included to aid in understanding the invention but is not to be construed as limiting the scope of the present invention as defined by the embodiments appended hereto. Descriptions of conventional methods that do not aid in understanding the present invention have not been included. Such methods are well known to those of ordinary skill in the art and are described in numerous publications. All references mentioned herein are incorporated in their entirety.

What is claimed is:

1. A process for increasing ratio of 4S to 4R sertraline stereoisomers in a mixture comprising isomerizing sertraline stereoisomers in a mixture having at least one sertraline 4R stereoisomer in the presence of a base and a catalytic amount of a solvent selected from the group consisting of: DMSO, tetrahydrofuran, methyl t-butyl ether, dioxane, sulfolane, N-methyl pyrrolidone and mixtures thereof.

2. The process of claim 1, wherein the solvent is added in an amount of about 0.02 ml/gr to about 0.2 ml/gr of the sertraline stereoisomers mixture.

3. The process of claim 2, wherein the solvent is added in an amount of about 0.05 ml/gr to about 0.2 ml/gr of the sertraline stereoisomers mixture.

4. The process of claim 1, wherein the base is an inorganic base.

5. The process of claim 4, wherein the inorganic base is selected from the group consisting of: an alkali metal, alkaline earth metal hydroxide, and a carbonate.

6. The process of claim 5, wherein the alkali metal is selected from the group consisting of: Na, K, Li, Mg, Ca and Ba.

7. The process of claim 5, wherein alkaline earth metal hydroxide is selected from the group consisting of: NaOH, KOH, LiOH and Mg(OH)2.

8. The process of claim 5, wherein the carbonate base is selected from the group consisting of: sodium carbonate, potassium carbonate, sodium hydrogen carbonate, and potassium hydrogen carbonate.

9. The process of claim 4, wherein the inorganic base is NaOH.

10. The process of claim 4, wherein the inorganic base is KOH.

11. The process of claim 1, wherein the base is added in a catalytic amount.

12. The process of claim 11, wherein the base is added in an amount of about 0.1 to about 0.5 equivalents of the sertraline stereoisomers mixture.

13. The process of claim 12, wherein the base is added in an amount of about 0.27 equivalents of the sertraline stereoisomers mixture.

14. The process of claim 1, wherein the isomerization step is performed at a temperature of about 80° C. to about 150° C.

15. The process of claim 14, wherein the isomerization step is performed at a temperature of about 100° C. to about 140° C.

16. The process of claim 15, wherein the isomerization step is performed at a temperature of about 110° C. to about 135° C.

17. The process of claim 1, wherein the isomerization process results in a ratio of about 1.1 to about 1.50 4R to 4S.

18. The process of claim 1, wherein the reduction in amount of 4R isomer is from at least about 8 to about 1.5 in relation to the 4S isomer.

19. The process of claim 18, wherein the reduction in amount of 4R isomer is from at least about 11 to about 1.1 in relation to the 4S isomer.

20. The process of claim 1, wherein the solvent is DMSO.

21. The process of claim 1, wherein the solvent is tetrahydrofuran.

22. The process of claim 1, wherein the solvent is methyl t-butyl ether.

23. The process of claim 1, wherein the solvent is dioxane.

24. A process for preparing sertraline HCl comprising isomerizing sertraline stereoisomers in a mixture having at least one sertraline 4R stereoisomer in the presence of a base and a catalytic amount of a solvent selected from the group consisting of: DMSO, tetrahydrofuran, methyl t-butyl ether, dioxane, sulfolane, N-methyl pyrrolidone and mixtures thereof, and converting the sertraline to sertraline HCl.

25. A process for increasing ratio of 4S to 4R sertraline stereoisomers comprising isomerizing sertraline stereoisomers in a mixture having at least one sertraline 4R stereoisomer in the presence of a solvent selected from the group consisting of DMSO, tetrahydrofuran, methyl t-butyl ether, dioxane, sulfolane, N-methyl pyrrolidone and mixtures thereof, and a catalytic amount of base.

26. The process of claim 25, wherein the base is added in about 0.1 to about 0.5 equivalents of the sertraline stereoisomers mixture.

27. The process of claim 26, wherein the base is added in an amount of about 0.27 equivalents of the sertraline stereoisomers mixture.

28. The process of claim 25, wherein the base is an inorganic base.

29. The process of claim 28, wherein the inorganic base is selected from the group consisting of: an alkali metal, alkaline earth metal hydroxide, and a carbonate.

30. The process of claim 29, wherein the alkali metal is selected from the group consisting of: Na, K, Li, Mg, Ca and Ba.

31. The process of claim 29, wherein alkaline earth metal hydroxide is selected from the group consisting of: NaOH, KOH, LiOH and Mg(OH)2.

32. The process of claim 29, wherein carbonate is selected from the group consisting of: sodium carbonate, potassium carbonate, sodium hydrogen carbonate, and potassium hydrogen carbonate.

33. The process of claim 28, wherein the inorganic base is NaOH.

34. The process of claim 28, wherein the inorganic base is KOH.

35. The process of claim 25, wherein the solvent is added in a catalytic amount.

36. The process of claim 35, wherein the solvent is added in an amount of about 0.02 ml/gr to about 0.2 ml/gr of the sertraline stereoisomers mixture.

37. The process of claim 36, wherein the solvent is added in an amount of about 0.05 ml/gr to about 0.2 ml/gr of the sertraline stereoisomers mixture.

38. The process of claim 25, wherein the isomerization step is performed at a temperature of about 80° C. to about 150° C.

39. The process of claim 38, wherein the isomerization step is performed at a temperature of about 100° C. to about 140° C.

40. The process of claim 39, wherein the isomerization step is performed at a temperature of about 110° C. to about 135° C.

41. The process of claim 25, wherein the isomerization process results in a ratio of about 1.1 to about 1.50 4R to 4S.

42. The process of claim 25, wherein the reduction in amount of 4R isomer is from at least about 8 to about 1.5 in relation to the 4S isomer.

43. The process of claim 42, wherein the reduction in amount of 4R isomer is from at least about 11 to about 1.1 in relation to the 4S isomer.

44. The process of claim 1, wherein the isomerization is carried out in a suspension

45. A process for preparing sertraline HCl comprising isomerizing sertraline stereoisomers in a mixture having at least one sertraline 4R stereoisomer in the presence of a solvent selected from the group consisting of DMSO, tetrahydrofuran, methyl t-butyl ether, dioxane, sulfolane, N-methyl pyrrolidone and mixtures thereof, and a catalytic amount of base, and converting the sertraline to sertraline HCl.

46. A process for oxidizing an isomerized mixture of sertraline stereoisomers comprising: combining an isomerized mixture of sertraline stereoisomers, a mixture of C1-C4 alcohol and alkyl aromatic hydrocarbon, and an oxidizing agent in the presence of a base.

47. The process of claim 46, wherein the C1-C4 alcohol is methanol.

48. The process of claim 46, wherein the alkyl aromatic hydrocarbon is toluene.

49. The process of claim 48, wherein the alcohol is methanol, and the ratio between the methanol to toluene is of about 5 to about 0.5 volume/volume.

50. The process of claim 46, wherein the oxidizing agent is selected from the group consisting of: KMnO4, Br2 and hypohalite, such as hypochloride.

51. The process of claim 46, wherein the base is an inorganic base.

52. The process of claim 5 1, wherein the inorganic base is selected from the group consisting of: an alkali metal, alkaline earth metal hydroxide, and a carbonate.

53. The process of claim 52, wherein the alkali metal is selected from the group consisting of: Na, K, Li, Mg, Ca and Ba.

54. The process of claim 52, wherein the alkaline earth metal hydroxide is selected from the group consisting of: NaOH, KOH, LiOH and Mg(OH)2.

55. The process of claim 52, wherein the carbonate is selected from the group consisting of: sodium carbonate, potassium carbonate, sodium hydrogen carbonate, and potassium hydrogen carbonate.

56. The process of claim 52, wherein the inorganic base is KOH.

57. The process of claim 52, wherein after combining the isomerized mixture of sertraline stereoisomers, a mixture of C1-C4 alcohol and alkyl aromatic hydrocarbon, and an oxidizing agent in the presence of a base, a slurry is obtained.

58. The process of claim 57, wherein the slurry is maintained for about 0.5 to about 12 hours.

59. The process of claim 59, wherein the obtained product is further recovered.

60. A process for preparing sertraline comprising: combining an isomerized mixture of sertraline stereoisomers, a mixture of C1-C4 alcohol and alkyl aromatic hydrocarbon, and an oxidizing agent in the presence of a base to oxidize the isomerized mixture, and reducing the oxidized mixture to obtain sertraline.
